(19) United States
(12) Patent Application Publication
(10) Pub. No.: US 2006/0236564 A1
(43) Pub. Date: Oct. 26, 2006

(54) Orthotic with dynamically self-adjusting stabiliser for footwear

(75) Inventors: Paul Allard, Montreal (CA); Sebastien Garcia, Velleron (FR); Sebastien Hinse, Montreal (CA)

Correspondence Address: LESPERANCE & MARTINEAU, 1440 WEST, STE-CATHERINE ROOM 700, MONTREAL, QC, H3G1R8, CA

(73) Assignees: CRYOS TECHNOLOGIES INC., N.D.P. Joliette (CA); UNIVERSITE DE MONTREAL, Montreal (CA)

(21) Appl. No.: 11/111,909

(22) Filed: Apr. 22, 2005

(51) Int. Cl. A61F 5/14

(52) U.S. Cl. 36/140

(57) ABSTRACT

The orthotic is formed of a monolithic semi-rigid resilient shell for engagement inside a footwear and for conformingly fitting against the plantar portion of a patient's foot. The orthotic includes a rear heel cup portion that may have inner and outer rearwardly-extending resiliently deformable arms defining at their rear ends a rearwardly-opened notch therebetween. A number of first grooves may be made along the outer or inner flange of the heel cup portion, on the exposed top or bottom surface thereof. The orthotic further comprises a front end portion for engaging the metatarsal plantar region of the foot, having a sinuous front edge which includes five frontwardly-facing arcuate concavities each for registering wedging engagement therein by a corresponding one of the five metatarsal anterior portions of that person's foot. A number of second grooves may be made on the exposed top or bottom surface of the front end portion, and at least one of each second groove open into a corresponding arcuate concavity. The front and rear grooves provide dynamic stabilization of the patient's foot and resistance against cyclical load-induced torque during gait. No separate add-on crutch stabilizer is necessary at a raised portion of the orthotic.

### FIELD OF THE INVENTION

The present invention relates to shoe inserts used by podiatrists in correcting a medical condition deficiency in the foot

### BACKGROUND OF THE INVENTION

Footwear insoles, also called podiatric orthotic or orthotic shoe inserts, are used to support the human foot in a footwear, and have been known for quite some time in the field. These devices consist usually of a moulded blank shell formed monolithically from a resilient semi-rigid sheet material, usually a synthetic plastic material. In some orthotic shoe inserts, the orthotic rear end portion may comprise an inner and an outer rearwardly-extending resiliently deformable arms defining a rearwardly-opened notch therebetween—the so-called heel cup. This heel cup, being raised above the sole level of the footwear, requires external semi-rigid cushion pads forming a crutch-like support that engage the footwear sole, to stabilize the heel portion of the foot against load-induced rotational torque during cyclical gait movement.

Orthotics are characterized by the fact that they properly adjust the orientation of a person's deficient foot during gait, for controlling its motion in view of mitigating the adverse effects of podiatric anomalies.

Problems associated with these known orthotic devices include:
(a) they take too much volume in the footwear, so the foot is uncomfortably compressed inside the footwear; and/or the orthotic cannot fit inside the footwear.
(b) they often are undesirably allowed to accidentally shift in position inside the footwear, especially during prolonged gait.
(c) they generate a second movement of the foot, to counteract the first medically deficient movement of the foot, and thus bring about instability of the orthotic under load.
(d) There are problems associated both with prolonged use and with maintenance of the conventional orthotic devices.
(e) Lesions can be created on the front plantar portion, under the metatarsal heads or rearwardly near them, by the front edge of the orthotic or of the insole. Indeed, the orthotics or insoles often extend short of the toes. Although the orthotic is expected to gradually frontwardly slope towards the shoe's sole so as to form therewith an almost continuous surface under the metatarsal head region, the reality is otherwise: the frontmost edge of the orthotic often repetitively raises during gait spacedly above the shoe's sole, and under repetitive contact with the foot plantar surface, is likely to cause lesions and injure the foot plantar metatarsal region. Moreover, the foot is often not allowed to recover from these lesions, since the already injured plantar foot portion continues to suffer lesions from its subtle contact with the linear front edge portion of the orthosis under continued use of the orthotic.

Some orthotics include grooves made on the upper and/or exposed lower surfaces thereof, which respectively control accidental slippage movement of the foot across the orthotic, and of the orthotic relative to the footwear insole. The purpose of these grooves in such latter known orthotics is thus to control the undesirable relative movements of the orthotics.

Other orthotics may have shock absorbing ribs at the rear end portion thereof, on their exposed surface. A problem with such orthotics is that they tend to create an external rotational force on the heel which causes heel supination at the tibio-fibular leg unit. These grooves operate as rotors that contribute in the rotation and the progressive shift in the position of the longitudinal axis of the orthotics. The orthotic is then used to generate a movement to counteract another movement, i.e. to generate supination to counteract pronation. Such orthotics will not limit the deviation of the segment or to correct its position, but rather will tend to control the internal rotational torque operating at the level of the tibio-femoral assembly.

### OBJECTS OF THE INVENTION

An object of the invention is to provide unitary orthotic device that will provide self-stabilizing control of its axial deviation from saggital plane under load.

An object of the invention is to provide a unitary orthotic device that will not require an external crutch-like stabilizer pad at the heel cup area

A general object of the present invention is to increase the convenience, performance, and ease of use to the patient of such orthotics.

An object of the invention is to reduce the overall volume of such orthotics, to enable use of this orthotics inside a more compact footwear.

A general object of this orthotic is to increase maximal operational lifetime thereof by improving upon the long term operational mechanical performance thereof during cyclical gait operation.

An important object of the orthotic is to generate a force of resistance against the foot so as to control the segmental deviation thereof and to correct a postural misalignment thereof, without generating rotational movement of the orthotic.

A more specific important object of the present orthotic is to improve the efficiency of posture correcting devices by providing an orthotic device generating an improved foot posture concurrently with better flow transfer of dynamic loading forces operating on the foot and on the lower leg section of the patient.

A further object of the invention is that the heel itself will stabilize the orthotic in position during gait, against load-borne shifts in position thereof inside the footwear.

### SUMMARY OF THE INVENTION

The invention consists of an orthotic device for engagement inside a footwear and for conformingly fitting against the plantar portion of a patient's foot for compensating podiatric deficiencies, said orthotic device being formed of a monolithic semi-rigid resilient shell defining inner and outer lateral sides, a top exposed surface and a bottom surface, and having: an arched intermediate portion for complementary vertical resilient spring-back engagement against the foot arch plantar portion; a rear end portion for receiving the patient's heel; a front end portion integrally frontwardly extending from said intermediate portion and for complementary engagement near the anterior metatarsal plantar region of the foot; and reinforcement means, integral to said shell, wherein said reinforcement means provides both dynamic stabilization at said shell front end portion and resistance against cyclical load-induced torque during gait at said rear end portion thereof.

In a first embodiment, said reinforcement means may be an integral part of said front end portion of the shell, or alternately in another embodiment, may be an integral part of said rear end portion of the shell, or in still another embodiment an integral part of both said front and rear portions of the shell. Said reinforcement means may include a number of grooves made on said top exposed surface of the shell, or alternately on said bottom surface thereof, or still alternately in a channel made within the shell itself in embedded, at least partly concealed fashion. Each of said grooves/channel may be cross-sectionally U-shaped. The depth of any one of each of said grooves/channels may be at least 1 mm and up to 2 mm in depth.

Said stabilization of the patient's foot brought about by said reinforcement means may be of the lateral type, and/or of the medial type, depending on the medical condition of the patient's.

In one embodiment, grooves/channels extend longitudinally of the shell; in alternate embodiments, grooves/channels extend transversely thereof, preferably at a small acute angle relative to the longitudinal axis of said shell.

### BRIEF DESCRIPTION OF THE DRAWINGS

In the annexed drawings:

FIG. 1 is a perspective view of a first embodiment of left-foot orthotic according to the invention;

FIGS. 2 and 3 are respectively a top plan view and an outer side edge elevation of the orthotic of FIG. 1;

FIG. 4 is a view similar to FIG. 2 but at a smaller scale;

FIGS. 5 and 6 are enlarged cross-sectional views taken along lines AA and BB respectively of FIG. 4; and

FIG. 7 is a perspective view of a second embodiment of orthotic device of the invention, showing grooves at the intermediate arch portion thereof.

### DETAILED DESCRIPTION OF THE INVENTION

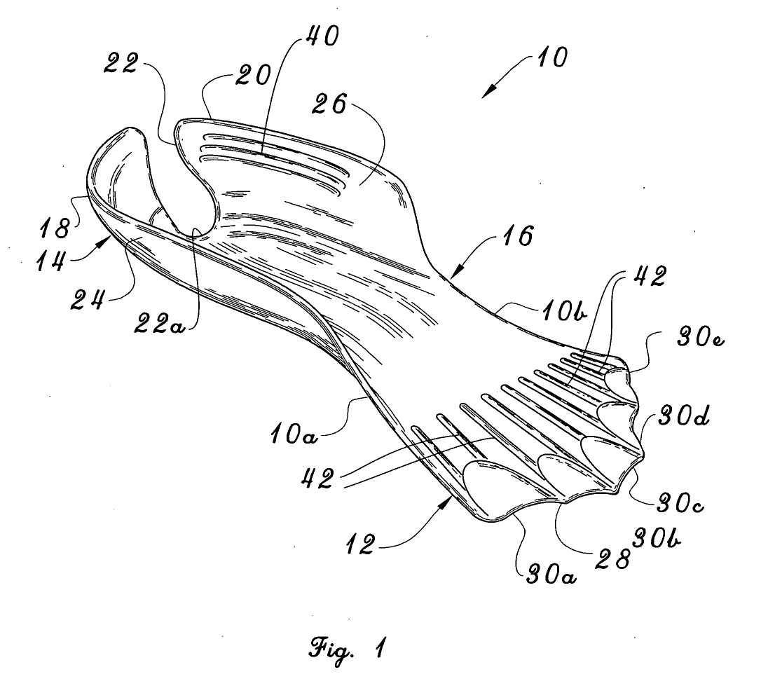

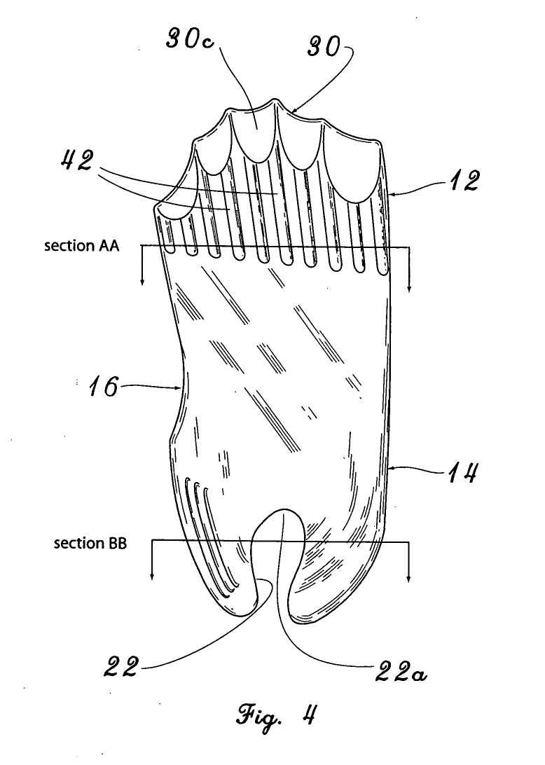

FIGS. 1 to 4 show an orthotic 10 according to the invention, being formed of a monolithic semi-rigid resilient shell and defining inner and outer lateral sides 10a, 10b, a front end portion 12, a rear end portion 14, and an intermediate portion 16 therebetween. Orthotic 10 is to be inserted inside a footwear (not shown) in a conventional fashion, for conformingly fitting against the plantar portion of a person's foot. The orthotic 10 shown in FIGS. 1-4 is a left-foot orthotic; however, any one of a left foot or a right foot orthotic may benefit from the present invention. Each orthotic 10 is usually custom-moulded to each foot, so as to precisely correspond to the foot of each individual. It is understood, for clarity of the present specification, that reference to the inner and outer lateral sides 10a, 10b of orthotic 10 will be made according to a person's feet, i.e. a person wearing a pair of right and left hand side orthoses 10, 10 will have their inner sides 10a, 10a facing each other, while their outer sides 10b, 10b will face outwardly in opposite directions. The inner side 10a of orthotic 10 has a generally convex shape and provides corrective support as known in the art, for example against hyperpronation; while the outer side 10b of orthotic 10 has a generally concave shape for occupying less volume inside the footwear and thus enhance comfort of the person wearing orthotic 10.

The intermediate portion 16 of orthotic 10 (between fore end 12 and aft end 14) is arched for complementary vertical engagement against the foot plantar arch portion. Moreover, under the resiliency of orthotic 10 and due to the arched shape of intermediate portion 16, the latter will have a positive substantially vertical spring-back effect on the foot arch portion during gait, for promoting corrective foot positioning.

In one embodiment of orthotic device, the orthotic rear end portion 14 comprises an inner and an outer rearwardly-extending resiliently deformable arms 18, 20 defining a rearwardly-opened notch 22 therebetween. Notch 22 defines an inner bottom edge 22A. Upwardly arcuate flanges 24, 26 are formed at the periphery of the orthotic rear end portion 14, with inner and outer arms 18, 20 respectively carrying the rear part of inner and outer flanges 24, 26. Flanges 24, 26 diverge from notch 22 and are both curved frontwardly as they extend up to intermediate portion 16, along the orthotic sides 10a, 10b. Inner flange 24 gradually blends into the orthotic inner arched intermediate portion 16 to form a substantially continuous inner edge 10a, while outer flange 26 is cut away frontwardly of rear end portion 14 of orthotic 10. Rear end portion 14, with arcuate flanges 24, 26, thus forms a cup-like seat for a person's heel, as will be explained hereinafter. Open notch 22, extends between flanges 24, 26 in an upwardly and outwardly curved fashion. Flange 24 controls pronation of foot, while flange 26 controls supination thereof.

In accordance with an important feature of the present invention, a number of grooves—for example three grooves 40 as shown are made on the upper exposed surface of flange 26 of outer deformable arm 20 of the heel cup. Grooves 40 are generally arcuate and parallel to one another in closely spaced fashion, and preferably of similar length relative to one another, with the outermost groove 40′ being spacedly proximate the edge 26A of flange 26. The rearmost end of each groove 40 extends in closely spaced fashion relative to notch 22, while the foremost end of each such groove 40 extend slightly beyond the level of inner bottom edge 22A of notch 22.

It has been unexpectedly found that the heel cup grooves 40, by reinforcing the heel cup rigidity, provide a dynamically enhancing reinforcement means that has the surprising result of allowing a patient to use his orthotic without an additional add-on component being the standard crutch-like semi-rigid external stabilizer pad. Indeed, the external stabilizer pad does not need to be added to the heel cup area of the present orthotic, since the heel cup design already incorporates an integral compensating means, namely, the grooves 40. Grooves 40 unexpectedly and most advantageously maintain and improve upon the orthotic mechanical performance under cyclical loading during gait, without increasing the structural volume nor weight thereof. This also means that the present orthotic 10 may further fit inside shallow compact footwear, where it could only fit with difficulty in the past because of the added volume brought about by the add-on external stabilizer pad (that worked as a crutch).

Front end portion 12 integrally frontwardly extends from intermediate portion 16 and becomes wider to conform to the shape of the foot anterior metatarsal region. Front end portion 12 has a sinuous front edge 28 comprising alternating apexes and recesses, to form a number of frontwardly-facing arcuate concavities generally referred to with number 30, and more particularly five concavities 30a, 30b, 30c, 30d and 30e as shown in the drawings. Front edge 28 is generally frontwardly convex so as to conform to the forefoot configuration.

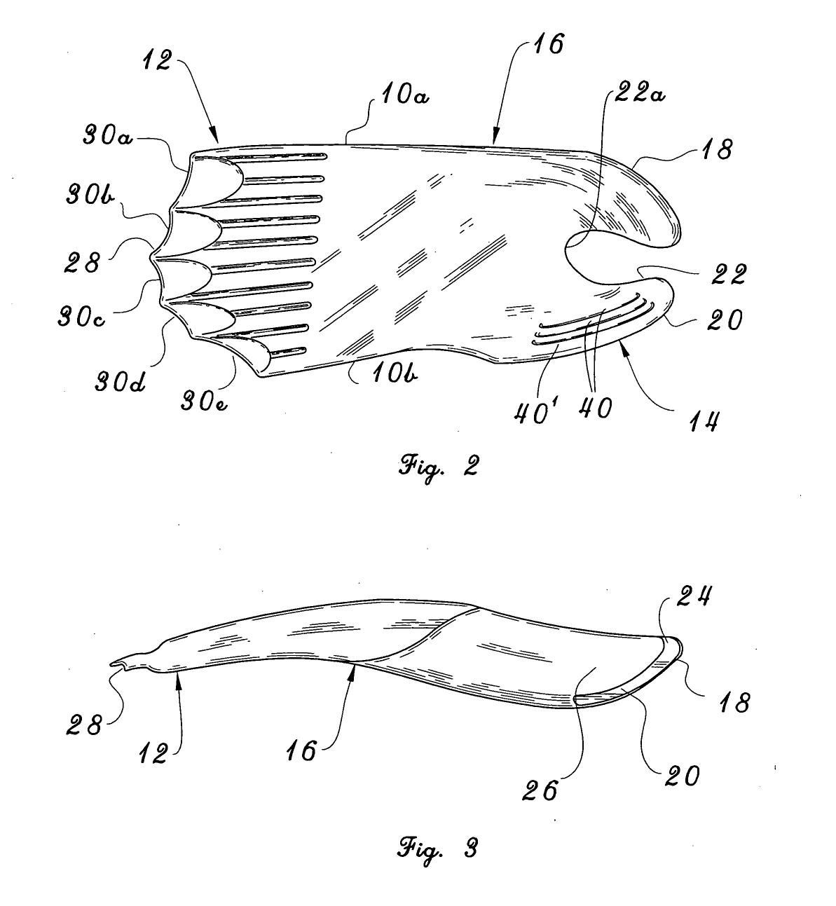

Another important feature of the present invention is the provision of a number of additional grooves—for example ten grooves 42 as shown—made on the upper exposed surface of front end portion 12 of orthotic 10. As best shown in FIG. 2, grooves 42 are generally divergent to one another, being disposed in fan-like fashion, with their inner ends all starting at about the same inner edge section of front end portion 12, and equally spread apart successively from side edge 10a to side edge 10b. One or more successive grooves 42 open into a corresponding coextensive concavity 30a, 30b, 30c, 30d or 30e, so that each said concavity is engaged by at least one such groove 42. Accordingly, the effective length of each groove 42 will vary depending upon where the outer (front) end of the groove 42 engages the concavity 30a-30e, and upon where the inner side of the foot is (the interior front end side of the orthotic will be longer than the exterior front end side, due to the anatomy of the foot).

For best performance, each groove 42 preferably has at least 1.0 millimeter (mm) in depth and 1.5 mm in width, although other sizes could also be effective; while the length thereof being variable from patient to patient in view of the overall size and medical condition of the patient's foot. The depth and width of interior concavity 30a should usually be greater than that of exterior concavity 30e.

Each heel groove 40 is preferably slightly larger and deeper than any one metatarsal groove 42. Each groove 40 could be for exemple 2 mm in depth and 2 mm in width

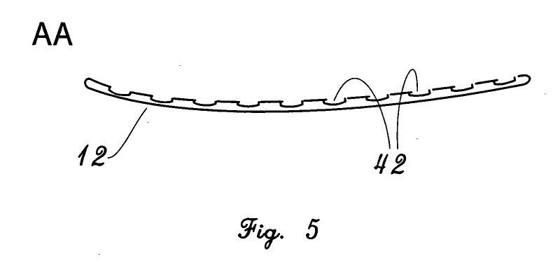

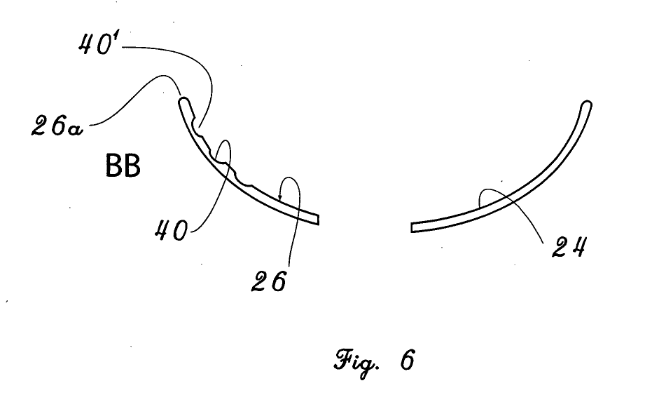

As shown in FIG. 5, each metatarsal groove 42 may form a square U-shape in cross-section. As also shown in FIG. 6, each heel groove 40 may form an ovoid in cross-section. However, it is understood that the metatarsal grooves 42 could alternately form an ovoid in cross-section, and similarly, each heel groove 40 could alternately form a square U-shape in cross-section. Also, grooves 40 and 42 could both have the same cross-sectional pattern, i.e. U-shape, ovoidal, or other suitable shape.

In use, orthotic 10 is to be positioned inside a footwear (not shown) against the plantar portion of the foot, with the intermediate portion 16 conformingly engaging the underface of the foot arch, as explained hereinabove. The orthotic rear portion 14 engages the foot heel portion, with partial engagement of the heel inside notch 22. The anterior portion of the foot calcaneum overlies orthotic 10 frontwardly relative to notch 22. Flanges 24, 26 complementarily engage the heel portion of the foot so as to partly surround heel so as to form a cup-like seat for heel.

Thus, positional self-adjustment of the orthotic is accomplished during gait by the load of the person being applied on his heel and against the orthotic arms 18, 20, which are yieldingly resiliently moved apart and pressed against the footwear inner walls to correctly re-position orthoric 10 in the footwear if load-induced temporary displacement of orthotic 10 occurred. Flanges 24, 26, in addition to promoting positional self-adjustment of orthotic 10 in the footwear, also provide positive foot support to enhance corrective foot positioning in the footwear.

The presence of notch 22 allows arms 18, 20 to be formed, with the above-mentioned advantages. Moreover, notch 22 allows a thinner orthotic rear portion configuration, since heel is effectively allowed to partly engage notch 22, and thus a thinner rear portion of orthotic 10 is located between heel and the footwear sole inner wall. Consequently, orthotic 10 is less cumbersome inside the footwear, hence a greater comfort for the person wearing orthotic 10.

The front end portion 12 of orthotic 10 is located near and complementarily engages the anterior metatarsal plantar region of the foot. More particularly, the orthotic front end portion extends just short of the metatarsal anterior portions or heads, with concavities 30 being positioned for registering engagement against a corresponding metatarsal anterior portion. Concavities 30a, 30b, 30c, 30d and 30e register respectively with the first, second, third, fourth and fifth metatarsal anterior portions. This allows distinct support and articulate positional self-adjustment of orthotic 10 in the footwear, since any deviation of the orthotic inside the footwear will be corrected by the load-bearing seating abutment of the metatarsal anterior portions against their respective concave orthotic recesses 30. Each metatarsal head bearing against a concavity 30 will effectively forcibly slide against the concave surface thereof under load-bearing conditions to properly align orthotic 10, hence further positional self-adjustment of orthotic 10 in the footwear. Generally, orthotic 10 will be less likely to shift inside the footwear due to the five metatarsal heads bearing against and being wedged in the corresponding number of concavities 30 under the load of the person wearing orthotic 10 in his footwear.

Moreover, concavities 30 additionally help prevent plantar lesions that often occur under prolonged use of a footwear orthotic. Indeed, concavities 30 provide a configuration to the orthotic front end portion 12 which is more adapted to the pressure points of the foot, i.e. that the metatarsal anterior portions will effectively be conformingly seated against their respective concavities 30 during load-bearing conditions, such as at toe-off. Prior art orthotics often provided straight front edges, that undesirably could, at length, cause lesions to the registering foot plantar surface under load-bearing conditions due to the abutment of the metatarsal anterior portions onto the orthotic straight front edges. The presence of concavities 30 helps to prevent and obviate this problem.

The unexpected results and important advantages of the orthotic of the present invention over the prior art orthotic are brought about by its particular structural characteristics, namely, that the self-adjustable stabilizing properties of the orthotic under load brought about by the exposed surface grooves 40, 42, made along the metatarsal region 12 and/or the heel portion 26 of the orthotic 10, without bringing about compensatory movement of the orthotic. The present orthotic device thus reinforces the shell structure in the lengthwise direction of the grooves, while providing flexibility to the shell transversely to the grooves. This is contrary to active compensation means being provided in prior art, that counteracts load induced rotational torque on the orthosis by transforming this movement into a compensatory one, thus bringing about instability due to shifts in center of gravity locus; wherein the function of the foot is undesirably locked. It is noted that such grooved areas 12, 26, of orthotic should be stronger in resistance loading than a non-grooved area.

The orthotic 10 may be manufactured for example under pressure molding with a polynyolene plastic sheet. The most simple way of generating the grooves 40 and 42 could be to form same by rigid cylindroid wires being applied against the plastic sheet, before the pressure molding operation. The orientation of metatarsal grooves 42 relative to the fore and aft lengthwise axis of orthotic 10, may vary according to the medical condition of the patient. For example, if the main medical constraint relates to foot flexing, then the wires will be oriented longitudinally to the orthotic. The rigidity of the orthotic, and thus its resistance to load-induced deformation during gait, can be modulated by varying the number of such grooves 40, 42, and the depth and width of each of them.

It is understood that the dynamic self stabilization of the patient's foot with the present orthotic is brought about by an increased mechanical resistance and structural integrity due to the introduction of grooves on a fraction of the upper face or bottom face of the orthotic 10, and in particular along the heel cup portion, and/or also about the metatarsal portion of this orthotic. Different groove orientations are considered relative to the longitudinal axis of the orthotic 10 depending on the foot deficiency to be treated. This dynamic stabilization corrects the foot position and reduces the segmental deviations. The addition of grooves does not recycle or reuse the biasing force generated during cyclical load gait, but rather introduce a resistance force applied to the foot in view of limiting segmental deviation and correcting postural misalignment, while maintaining flexibility to the foot in other respects.

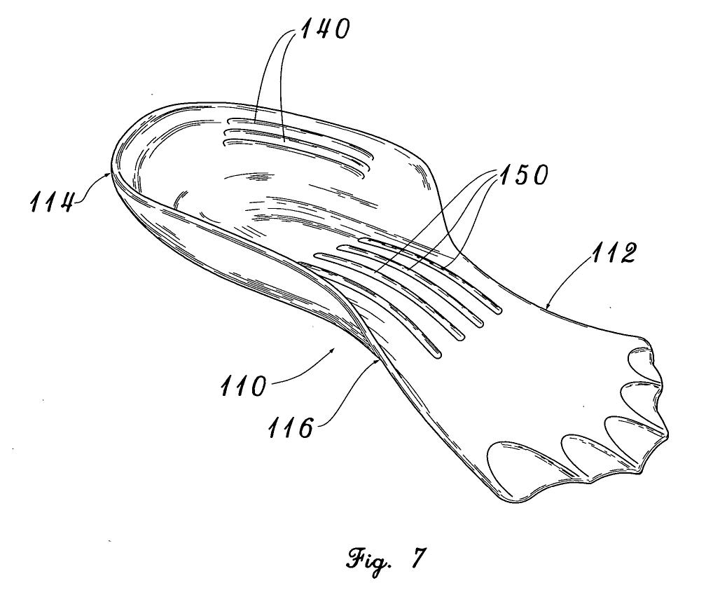

FIG. 7 shows an alternate embodiment of orthotic 110, having a series of grooves 150 made lengthwisely about the intermediate arch portion 116 of orthotic 110. Heel portion 114 of orthotic 110 is devoid of notch (22 in the embodiment of FIG. 1), but may have grooves 140. No groove is shown at the front end portion 112 of orthotic 110.

Any modifications to the present invention, which do not deviate from the scope thereof, are considered to be included therein. For example, the network of grooves 40 and/or 42 could be replaced by a corresponding wavy surface. Also, grooves 40, 42, need not be parallel to one another, and may for example be divergent from one another in fan-like arrangement as would be the case to complement the metatarsal portion of the front end portion of the patient's foot. Grooves or channels could alternately be filled with an embedded graphite wire, a fibreglass wire, or the like wire structure remaining embedded into the orthotic device shell.

I claim:

1. An orthotic device for engagement inside a footwear and for conforminglyfitting against the plantar portion of a patient's foot for compensating podiatric deficiencies, said orthotic device being formed of a monolithic semi-rigid resilient shell defining inner and outer lateral sides, a top exposed surface and a bottom surface, and having: an arched intermediate portion for complementary vertical resilient spring-back engagement against the foot arch plantar portion; a rear end portion for receiving the patient's heel; a front end portion integrally frontwardly extending from said intermediate portion and for complementary engagement near the anterior metatarsal plantar region of the foot; and reinforcement means, integral to said shell, wherein said reinforcement means provides both dynamic stabilization at said shell front end portion and resistance against cyclical load-induced torque during gait at said rear end portion thereof.

2. An orthotic device as in claim l,
wherein said reinforcement means is an integral part of said front end portion of the shell.

3. An orthotic device as in claim 1,
wherein said reinforcement means is an integral part of said rear end portion of the shell.

4. An orthotic device as in claim 1,
wherein said reinforcement means is an integral part of both said front end portion and of said rear end portion of the shell.

5. An orthotic device as in claim 1,
wherein said reinforcement means includes a number of grooves made on said top exposed surface of the shell.

6. An orthotic device as in claim 1,
wherein said reinforcement means includes a number of grooves made on said bottom surface of the shell.

7. An orthotic device as in claim 1,
wherein said reinforcement means includes a number of grooves made on both said top exposed surface and of said bottom surface of the shell.

8. An orthotic device as in claim 1,
wherein said reinforcement means includes a number of channels made within the shell itself in embedded, at least partly concealed fashion.

9. An orthotic device as in claim 5,
wherein each of said grooves is cross-sectionally U-shaped.

10. An orthotic device as in claim 5,
wherein the depth of each of said grooves is at least 1.5 mm.

11. An orthotic device as in claim 3,
wherein said reinforcement means includes a number of grooves made on said top exposed surface of said rear end portion of the shell, the depth of each of said grooves being at least 2 mm in depth.

12. An orthotic device as in claim 5,
wherein said grooves extend generally longitudinally of the shell.

13. An orthotic device as in claim 5,
wherein said grooves extend transversely and at a small acute angle relative to the longitudinal axis of said shell.

14. An orthotic device as in claim 2,
wherein said reinforcement means includes a number of grooves made on said top exposed surface of said front end portion of the shell.

15. A n orthotic device as in claim 14,
wherein said grooves at said front end portion of the shell extend in a forwardly diverging fan-like fashion.

16. An orthotic device as in claim 1,
wherein said reinforcement means includes a number of channels made within said shell.
